(19) United States
(12) Patent Application Publication
(10) Pub. No.: US 2023/0338532 A1
(43) Pub. Date: Oct. 26, 2023

(54) IMMUNOSUPPRESSANT DRUG RESISTANT ARMORED TCR T CELLS FOR IMMUNE-THERAPY OF ORGAN TRANSPLANT PATIENTS

(71) Applicant: NATIONAL UNIVERSITY OF SINGAPORE, SINGAPORE (SG)

(72) Inventors: Antonio BERTOLETTI, Singapore (SG); Anthony Tanoto TAN, Singapore (SG); Morteza HAFEZI, Singapore (SG)

(21) Appl. No.: 18/044,429

(22) PCT Filed: Sep. 10, 2021
(86) PCT No.: PCT/SG2021/050545

§ 371 Date: Mar. 8, 2023

Related U.S. Application Data
(60) Provisional application No. 63/077,034, filed on Sep. 11, 2020.

(51) Int. Cl. A61K 39/00; A61P 35/00; C07K 14/725; C12N 9/16; C12N 9/04

(52) U.S. Cl. CPC A61K 39/4632; A61K 39/4611; A61K 39/4622; A61K 39/464463; A61K 39/464454; A61K 39/464838; A61P 35/00; C07K 14/7051; C12N 9/16; C12Y 301/03016; C12N 9/0006; C12Y 101/01205; A61K 2239/53

(57) ABSTRACT

Described are novel immunosuppressant drug resistant armored (IDRA) T cells that co-express an exogenous T cell receptor (TCR) and one or more exogenous inhibitors of an immunosuppressant. The TCR can bind to an antigen expressed by a tumor cell or virally infected cell. Also described are methods of producing the modified T cell, and methods of treating a subject using the modified T cells.

### BACKGROUND OF THE INVENTION

Therapeutic strategies that harness the power of the immune system, by the adoptive transfer of T cells engineered to recognize cancer or virus-infected cells through introduction of specific CAR or TCR, are beginning to show efficacy. Such T cell therapies, however, are rarely utilized in patients with organ transplants, since the immunosuppressant regimens that are required to avoid organ rejection can suppress their function. The instant disclosure provides Immunosuppressant Drug Resistant Armored (IDRA) TCR T cells of desired specificity (including but not limited to HBV and EBV) that are transiently resistant to immunosuppressants. Such cells are useful for treating diseases occurring in patients receiving immunosuppressants. For example, while HBV-TCR T cells have shown anti-tumour efficacy in some liver transplanted patients with HCC recurrence, their effectiveness is limited by the immunosuppressant drugs administered to prevent liver graft rejection. IDRA HBV-TCR T cells can be used in this setting to enhance the in vivo function of the adoptively transferred TCR T cells. The IDRA TCR T cells can also be used to treat other common pathologies associated with immunosuppressant treatment, such as the reactivation of Epstein Barr virus or cytomegalovirus in patients receiving immunosuppressants after stem cell or organ transplantation.

### BRIEF SUMMARY OF THE INVENTION

Described herein are compositions and methods that are useful to engineer the specificity of T-cells and make them resistant to immunosuppressants. In one aspect, a modified T cell is described, the modified T cell comprising an exogenous inhibitor of an immunosuppressant and an exogenous T-cell receptor (TCR). Such modified T cells are sometimes referred to herein as Immunosuppressant Drug Resistant Armored (IDRA)-TCR T cells. In some embodiments, the modified T cell comprises an mRNA (e.g., a first mRNA) encoding an exogenous inhibitor of an immunosuppressant and an mRNA (e.g., a second mRNA) encoding an exogenous T-cell receptor (TCR). In some embodiments, the immunosuppressant is selected from Tacrolimus, Mycophenolate mofetil (MMF), or a combination thereof.

In some embodiments, the exogenous inhibitor is a mutant calcineurin (CN) subunit B (CnB) protein. In some embodiments, the mutant CnB is CnB30. In some embodiments, the mutant CnB is encoded by a nucleic acid sequence comprising SEQ ID NO:1 or SEQ ID NO:2, or a sequence having at least 90% sequence identity (e.g., at least 90%, 91%, 92%, 93%, 94%, 95%, 96%, 97%, 98%, or 99% sequence identity) to SEQ ID NO:1 or SEQ ID NO:2. In some embodiments, the mutant CnB protein comprises the amino acid sequence of SEQ ID NO:5, or a sequence having at least 90% sequence identity (e.g., at least 90%, 91%, 92%, 93%, 94%, 95%, 96%, 97%, 98%, or 99% sequence identity) to SEQ ID NO:5.

In some embodiments, the exogenous inhibitor is a mutant inosine 5′-monophosphate dehydrogenase (IMPDH) protein. In some embodiments, the mutant IMPDH protein is encoded by a nucleic acid sequence comprising SEQ ID NO:3 or SEQ ID NO:4 or a sequence having at least 90% sequence identity (e.g., at least 90%, 91%, 92%, 93%, 94%, 95%, 96%, 97%, 98%, or 99% sequence identity) to SEQ ID NO:3 or SEQ ID NO:4. In some embodiments, the mutant IMPDH protein comprises the amino acid sequence of SEQ ID NO:6, or a sequence having at least 90% sequence identity (e.g., at least 90%, 91%, 92%, 93%, 94%, 95%, 96%, 97%, 98%, or 99% sequence identity) to SEQ ID NO:6.

In some embodiments, the exogenous TCR specifically binds to a viral antigen selected from a hepatitis B virus (HBV) antigen, a CMV antigen, an EBV antigen, an influenza antigen, or a SARS antigen. In some embodiments, the exogenous TCR specifically binds to the HBV envelope 183-191 antigen, the HBV core 18-27 antigen, or the EBV-LMP2 antigen. In some embodiments, the exogenous TCR specifically binds to an antigen in Table 1.

In some embodiments, the T cell is isolated from a subject. In some embodiments, the subject has a liver disease. In some embodiments, the subject has received an organ transplant and is administered an immunosuppressant. In some embodiments, the subject additionally has a viral infection or a tumor. In some embodiments, the subject is immunocompromised.

In another aspect, a method for producing a modified T cell, e.g., an IDRA TCR T cell, is described, the method comprising introducing an mRNA encoding an exogenous inhibitor of an immunosuppressant and an mRNA encoding an exogenous TCR into the T cell. In some embodiments, the exogenous inhibitor is a mutant calcineurin (CN) subunit B (CnB) protein. In some embodiments, the mutant CnB protein comprises the amino acid sequence of SEQ ID NO:5, or a sequence having at least 90% sequence identity (e.g., at least 90%, 91%, 92%, 93%, 94%, 95%, 96%, 97%, 98%, or 99% sequence identity) to SEQ ID NO:5. In some embodiments, the exogenous inhibitor is a mutant inosine 5′-monophosphate dehydrogenase (IMPDH) protein. In some embodiments, the mutant IMPDH protein comprises the amino acid sequence of SEQ ID NO:6, or a sequence having at least 90% sequence identity (e.g., at least 90%, 91%, 92%, 93%, 94%, 95%, 96%, 97%, 98%, or 99% sequence identity) to SEQ ID NO:6. In some embodiments, the immunosuppressant is selected from Tacrolimus, Mycophenolate mofetil (MMF), or a combination thereof.

In another aspect, a method of treating disease in a subject who has been administered an immunosuppressant is described, the method comprising introducing a modified T cell, for example, an IDRA TCR T cell, described herein into the subject. In some embodiments, the disease is liver disease. In some embodiments, the liver disease is hepatocellular carcinoma (HCC). In some embodiments, the subject has previously received a liver transplant. In some embodiments, the subject has a viral infection, for example an HBV, CMV, EBV, influenza or SARS infection. In some embodiments, the subject has previously received an organ transplant. In some embodiments, the T cell is an autologous T cell.

In some embodiments, the immunosuppressant administered to the subject is selected from Tacrolimus, Mycophenolate mofetil (MMF), or a combination thereof.

In another aspect, a method of treating liver disease in a subject in need thereof is described, the method comprising introducing mRNA into a T cell isolated from the subject, wherein the mRNA encodes a mutant CnB protein, or the mRNA encodes a mutant IMPDH protein, or different mRNAs, where one mRNA encodes a mutant CnB protein and a second mRNA encodes a mutant IMPDH protein; and mRNA encoding an exogenous T-cell receptor, and reintroducing the T cell into the subject, wherein the subject is administered an immunosuppressant. Thus, in some embodiments, the mRNA introduced into the isolated T cell comprises different species of mRNAs or a plurality of different mRNAs, where one or a first mRNA encodes a mutant CnB protein, a different or second mRNA encodes a mutant IMPDH protein, and a different or third mRNA encodes an exogenous T-cell receptor. In some embodiments, the mutant CnB protein comprises the amino acid sequence of SEQ ID NO:5, or a sequence having at least 90% sequence identity (e.g., at least 90%, 91%, 92%, 93%, 94%, 95%, 96%, 97%, 98%, or 99% sequence identity) to SEQ ID NO:5. In some embodiments, the mutant IMPDH protein comprises the amino acid sequence of SEQ ID NO:6, or a sequence having at least 90% sequence identity (e.g., at least 90%, 91%, 92%, 93%, 94%, 95%, 96%, 97%, 98%, or 99% sequence identity) to SEQ ID NO:6.

In some embodiments, the mRNA is transiently expressed.

In some embodiments, the liver disease is hepatocellular carcinoma (HCC). In some embodiments, the subject has previously received a liver transplant. In some embodiments, the immunosuppressant is Tacrolimus, Mycophenolate mofetil (MMF), or a combination thereof.

In some embodiments of the method, the exogenous TCR specifically binds to a viral antigen selected from a hepatitis B virus (HBV) antigen, a CMV antigen, or an EBV antigen. In some embodiments, the exogenous TCR specifically binds to the HBV envelope 183-191 antigen, the HBV core 18-27 antigen, or the EBV-LMP2 antigen. In some embodiments, the exogenous TCR specifically binds to an antigen in Table 1.

### BRIEF DESCRIPTION OF THE DRAWINGS

FIGS. 1A and 1B show functional profiles of 5183-electroporated T cells treated with tacrolimus. Cytokine production (TNF-α as a representative) of drug-treated S183 TCR-T cells was evaluated following overnight co-culture with HepG2.215 cells. FIG. 1A: Concatenated dot plots (left panel) from representative experiments stained for TNF-α production. Bar graphs (right panel) demonstrate the percentage of cytokine-positive cells in 3 different healthy donors. Non-treated electroporated T cells used as negative control. FIG. 1B: Drug effect on T cell cytolysis determined through impedance measurement. The Normalized Cell Index plot was converted to an area under the curve, and quantified to measure the percentage of cytolysis ˜45 hours after S183-TCR T cell addition. Each dot represents one individual experiment in bar graphs. Statistical significance was evaluated by 2-tailed t test. (P-value: *0.01 to 0.05, **0.001 to 0.01, ***0.0001 to 0.001, ****<0.0001, n.s., not significant).

FIG. 2 shows a schematic representation of the Calcineurin pathway after T cell activation in the presence or absence of Tacrolimus. Antigen-mediated stimulation of the T cell receptor (TCR) results in Ca2+ signalling activation. Increasing cytoplasmic Ca2+ concentration subsequently activates serine and threonine phosphatase calcineurin, which dephosphorylates NFAT transcription factor. As a result, NFAT translocates to the nucleus and induces expression of T cell-associated genes including TNF-α, IFN-γ and IL-2. On the other hand, Tacrolimus complex with FKBP1A can interact with CnB subunit at calcineurin complex, which inhibits phosphatase activity and subsequent NFAT pathway activation.

FIGS. 3A, 3B and 3C show overexpression of mutant CnB does not impair s183 TCR expression and recovered T cell function in the presence of therapeutic concentrations of Tacrolimus. FIG. 3A: S183 TCR and mutant CnB m-RNA were co-electroporated and T cell function was evaluated following overnight co-culture with HepG2.2.15 cells. FIG. 3B: Viability and TCR expression of engineered T cells evaluated 24 hours post-electroporation. Non-electroporated (NEP) T cells used as a negative control in the experiments. FIG. 3C: Frequency of TNF-α-producing CD8+ cells out of total live CD8+ T cells were quantified following overnight incubation with the targets (n=3). Concatenated dot plots from representative experiments stained for TNF-α production. Non-electroporated T cells considered as control.

FIGS. 4A, 4B, and 4C show overexpression of mutant IMPDH* recover T cell viability in the presence of therapeutic concentration of MMF. FIG. 4A: Schematic representation of MMF signaling and effect on T cells. Active form of the drug, MPA, inhibits inosine 5′-monophosphate dehydrogenase (IMPDH) in the cytoplasm which is essential for de novo purine synthesis and selectively inhibits lymphocyte proliferation. FIG. 4B: Frequency of TNF-α-producing CD8+ cells out of total live CD8+ T cells were quantified following overnight incubation with the targets (n=3). Concatenated dot plots from representative experiments stained for TNF-α production. Non-electroporated T cells considered as control. FIG. 4C: Viability of IMPDH electroporated T cells evaluated 72 hours after exposure to clinically relevant concentration of MMF. (P-value: *0.01 to 0.05, **0.001 to 0.01, ***0.0001 to 0.001, ****<0.0001, n.s., not significant).

FIGS. 5A and 5B show FKBP12 si-RNA-mediated knockdown partially recovers T cell function in the presence of Tacrolimus. FIG. 5A: Sequence information of siRNA specific for FKBP1A. FIG. 5B: Frequency of TNF-α-producing CD8+ cells out of total live CD8+ T cells were quantified following overnight incubation with the targets and different concentrations of Tacrolimus (n=3). Concatenated dot plots from representative experiments stained for TNF-α production. Non-electroporated T cells considered as control.

FIGS. 6A, 6B and 6C show dual-resistant TCR-redirected T cells were produced by electroporating 3 mRNAs (HBV TCR, CnB mutant and IMPDH mutant) into the T cells. FIG. 6A: Frequency of TNF-α-producing CD8+ cells out of total live CD8 T cells was quantified following overnight incubation with the targets (n=3). Concatenated dot plots from representative experiments stained for TNF-α production. Non-treated mock electroporated T cells from same donor served as negative control. FIG. 6B: T cell cytolysis determined by real time killing assay in the presence and absence of both drugs. Bar graphs in the right panel demonstrate percentage of T cell cytolysis up to 45 hours after TCR-T cell addition to the targets. The lines in the graph in the left panel correspond to the treatments on the X-axis in the right panel of FIG. 6B. FIG. 6C: Viability of dual resistant TCR-T cells evaluated 72 hours after exposure to clinically relevant concentration of both drugs.

FIGS. 7A and 7B show engineering IDRA EBV-specific TCR-redirected T cells. FIG. 7A: IDRA EBV TCR-T cells were developed by electroporating m-RNA encoding EBV-specific TCR, mutant CnB and mutant IMPDH. Engineered T cells were co-incubated with HLA-A2+EBV-specific peptide pulsed (+) or non-pulsed (−) T2 cells overnight. Intracellular cytokine staining and viability analysis were performed at the indicated time after treatment. Concatenated dot plots from representative experiments stained for TNF-α. Bar graphs demonstrate the percentage of TNF-α-positive CD8+ T cells (n=3). Non-treated mock electroporated T cells from same donor served as negative control. FIG. 7B: Viability of dual resistant TCR-T cells evaluated 72 hours after exposure to clinically relevant concentration of both drugs.

### DEFINITIONS

Abbreviations: TCR stands for T cell receptor. CAR stands for chimeric antigen receptor.

The term “IDRA TCR T cell” as used herein refers to an immunosuppressant drug resistant armored T-cell that co-expresses an exogenous T cell receptor (TCR) and one or more exogenous inhibitors of an immunosuppressant.

As used herein, “activated T cell” refers to a T cell that expresses cytokines after binding of the TCR to an antigen presented by an antigen presenting cell (APC). The APC can present the antigen in the context of a MHC class I or class II molecule. In some embodiments, the APC can present the antigen in the context of a MHC class I molecules.

The term “exogenous” refers to a polynucleotide or protein that is not naturally present in a cell or not naturally present in a given context in the cell.

The term “comprising” is open ended and does not exclude other components, ingredients, or steps. Accordingly, the term “comprising” encompasses the more restrictive terms “consisting essentially of” and “consisting of.”

With the term “consisting essentially of” it is understood that the exogenous TCR polypeptide and/or polynucleotide “substantially” comprises the indicated sequence as an “essential” element. Additional sequences may be included at the 5′ end and/or at the 3′ end. Accordingly, a polypeptide “consisting essentially of” sequence X will be novel in view of a known polypeptide accidentally comprising the sequence X.

With the term “consisting of” it is understood that the polypeptide and/or polynucleotide according to the invention corresponds to at least one of the indicated sequences (for example a specific sequence indicated with a SEQ ID Number or a homologous sequence or fragment thereof).

The term “exogenous T cell receptor” (TCR) is herein defined as a recombinant TCR which is expressed in a cell by introduction of exogenous nucleic acid coding sequences for a TCR. In particular, the epitope-reactive TCR may be expressed in a cell in which the TCR is either not natively expressed or is expressed at levels that are insufficient to induce a response by the cell or a responder cell upon TCR-ligand binding.

The term “fragment” is herein defined as an incomplete or isolated portion of the full sequence of the antigen or epitope-reactive exogenous TCR which comprises the active site(s) that confers the sequence with the characteristics and function of the HBV epitope-reactive exogenous TCR. In particular, it may be shorter by at least one nucleotide or amino acid. The fragment comprises the active site(s) that enable the epitope-reactive exogenous TCR to recognise and bind to the epitope.

The term “HBV epitope-reactive T Cell Receptor (TCR)” is herein defined as a TCR which binds to an HBV epitope in the context of a Major Histocompatibility Complex (MHC) molecule to induce a helper or cytotoxic response in the cell expressing the recombinant TCR. In particular, the HBV epitope may be HBs 183-191, HBs 370-79 or HBc 18-27. More particularly, the HBV epitope may comprise the sequence of SEQ ID NO:25. The HBV epitope may be HBs 370-79. More particularly, the HBV epitope may comprise the sequence of SEQ ID NO:56, SEQ ID NO:57 or SEQ ID NO:58.

The term “HBc 18-27 epitope” is herein defined as an epitope that can stimulate HLA class I restricted T cells. It may be used interchangeably in the present invention as HBc18, HBc18-27, and HBc18-27 peptide. The sequence of the epitope may be “FLPSDFFPSV” (SEQ ID NO:25). In the present invention, the term HBc18-27 is used to refer to the HBc18-27 epitope of genotype A/D prevalent amongst Caucasians of sequence SEQ ID NO:25 unless otherwise stated. The region of the T cell receptor that binds to the epitope is referred to as HBc18-27 TCR or HBc18 TCR.

The term “HBs 370-79 epitope” is herein defined as an epitope that can stimulate HLA class I restricted T cells. The sequence of the epitope may be “SIVSPFIPLL” (SEQ ID NO:56). In the present invention, the term HBs 370-79 is used to refer to the HBs 370-79 epitope of genotype A/D prevalent amongst Caucasians of sequence SEQ ID NO:56 unless otherwise stated. The region of the T cell receptor that binds to the epitope is referred to as HBs370-79 TCR.

The term “immunotherapeutically effective amount” is herein defined as an amount which results in an immune-mediated prophylactic or therapeutic effect in the subject, i.e., that amount which will prevent or reduce symptoms compared to pre-treatment symptoms or compared to a suitable control.

The term “isolated” is herein defined as a biological component (such as a nucleic acid, peptide or protein) that has been substantially separated, produced apart from, or purified away from other biological components in the cell of the organism in which the component naturally occurs, i.e., other chromosomal and extrachromosomal DNA and RNA, and proteins. Nucleic acids, peptides and proteins that have been isolated thus include nucleic acids and proteins purified by standard purification methods. The term also embraces nucleic acids, peptides and proteins prepared by recombinant expression in a host cell as well as chemically synthesized nucleic acids.

The term “operably connected” is herein defined as a functional linkage between regulatory sequences (such as a promoter and/or array of transcription factor binding sites) and a second nucleic acid sequence, wherein the regulatory sequences direct transcription of the nucleic acid corresponding to the second sequence.

The term “mutant” or “mutant form” of a TCR epitope is herein defined as one which has at least one amino acid sequence that varies from at least one reference sequence via substitution, deletion or addition of at least one amino acid, but retains the ability to bind and activate the TCR bound and activated by the non-mutated epitope. In particular, the mutants may be naturally occurring or may be recombinantly or synthetically produced.

The term “subject” is herein defined as vertebrate, particularly mammal, more particularly human. For purposes of research, the subject may particularly be at least one animal model, e.g., a mouse, rat and the like. In particular, for the animal models, the sequence of the TCR.alpha.- and .beta.-chains may be selected based on species. In some cases, transgenic animals expressing human MHC molecules may also be useful in evaluating specific aspects of the present invention.

A person skilled in the art will appreciate that the present invention may be practiced without undue experimentation according to the method given herein.

The term “sequence identity” refers to two or more nucleic acid or amino acid sequences that are the same. Two or more nucleic acid or amino acid sequences can also share a certain percentage of nucleotides or amino acids that are the same, for example, at least 80%, 85%, 90%, 91%, 92%, 93%, 94%, 95%, 96%, 97%, 98%, 99% or 100% identity relative to a reference sequence over a specified region. The percentage of sequence identity can be determined by comparing two optimally aligned sequences over a comparison window. Sequence alignment methods are well known in the art, for example, the local homology algorithm of Smith and Waterman (Adv. Appl. Math. 2:482, 1970), the homology alignment algorithm of Needleman and Wunsch (J. Mol. Biol. 48:443, 1970), the search for similarity method of Pearson and Lipman (Proc. Natl. Acad. Sci. USA 85:2444, 1988), computerized implementations of these algorithms (e.g., GAP, BESTFIT, FASTA, and TFASTA in the Wisconsin Genetics Software Package, Genetics Computer Group, 575 Science Dr., Madison, Wis.), or manual alignment and visual inspection (see, e.g., Ausubel et al., Current Protocols in Molecular Biology (1995 supplement)). Additional algorithms include the BLAST and BLAST 2.0 algorithms, which are described in Altschul et al. (Nuc. Acids Res. 25:3389-402, 1977), and Altschul et al. (J. Mol. Biol. 215:403-10, 1990), respectively. Software for performing BLAST analyses is publicly available through the National Center for Biotechnology Information (see the internet at www.ncbi.nlm.nih.gov/). [0040] The terms “bind” and “specifically binds,” in the context of TCR specificity for an antigen or antigenic peptide, refers to the binding affinity between a TCR and a target antigen peptide bound to a major histocompatibility complex (MEC) molecule, and can be expressed as the dissociation constant (Kd) between a TCR and an antigenic peptide-MHC. The Kd can be in the range of 1-100 μM, with an association rate (kon) of 1000-10000 M⁻¹ s⁻¹ and a dissociation rate (koff) of 0.01-0.1 s⁻¹, as determined by surface plasmon resonance (SPR).

### DETAILED DESCRIPTION OF THE INVENTION

Described herein are compositions and methods that are useful to engineer the specificity of T-cells and make them resistant to immunosuppressants. In one aspect, described herein is a modified T cell that co-expresses an exogenous T cell receptor (TCR) and one or more exogenous inhibitors of an immunosuppressant. In some embodiments, the modified T cell is an immunosuppressant drug resistant armored T-cell that co-expresses an exogenous T cell receptor (TCR) and one or more exogenous inhibitors of an immunosuppressant (referred to as an IDRA TCR T-cell). In one aspect, described herein is a method for producing a modified T cell described herein, the method comprising (i) modifying a T cell to express T cell receptors (TCR) that specifically bind to an antigen expressed by a target cell, and (ii) modifying the T cell to confer resistance to an immunosuppressant, or reduce the activity of an immunosuppressant. In some embodiments, the TCR is an exogenous TCR that is not normally expressed by the T cell. In some embodiments, the antigen is expressed by a tumor cell. In some embodiments, the antigen is a peptide expressed by a virus. In some embodiments, the antigen is a peptide from HBV, EBV, or CMV. In some embodiments, the antigen is expressed by a cell infected with a virus.

In some embodiments, the method comprises an adoptive T-cell immunotherapy strategy where autologous T-cells isolated from the peripheral blood of HCC patients are modified to comprise (i) T cell receptors (TCR) that specifically bind HBV peptides presented on the surface of the HCC cells (HBV-TCR T-cells); and (ii) an agent that confers resistance to an immunosuppressant, or reduces the activity of an immunosuppressant. In some embodiments, the TCR is an exogenous TCR that is not normally expressed by the autologous T cell. In some embodiments, the agent comprises a molecule or compound that decreases expression of a gene or protein in an immunosuppressant pathway. In some embodiments, the agent comprises a nucleic acid that inhibits expression of an mRNA encoding a gene or protein in an immunosuppressant pathway. In some embodiments, the agent comprises a mutated version of the gene or protein. In some embodiments, the agent is overexpressed in the modified cell.

In some embodiments, the immunosuppressant is tacrolimus (FK506), and the immunosuppressant pathway is a pathway that activates the NFAT/NF-kappaB pathway or the calcineurin pathway. In some embodiments, the agent is a mutant calcineurin (CN) subunit B (CnB) protein.

In some embodiments, the agent inhibits tacrolimus binding protein FKBP1A. In some embodiments, the agent is si-RNA that decreases expression of FKBP1A mRNA and thus reduces the amount of FKBP1A protein expressed by the cell.

In some embodiments, the immunosuppressant is Mycophenolate mofetil (MMF). In some embodiments, the agent comprises a mutant IMPDH protein.

The methods and compositions described herein provide the following unexpected advantages.

First, for safety purposes, mRNA encoding antigen-specific T-cell receptors is introduced into T-cells such that the exogenous TCR is transiently expressed by the modified T cell. The method results in limiting the functional lifespan of the engineered T-cells to about 3-5 days in vivo, after which the modified T cells revert to non-specific autologous T-cells.

In contrast, current methods in the field of chimeric antigen receptor (CAR)/TCR T-cell immunotherapy have focused primarily on viral vector transduction methods to engineer T-cells that are able to stably express the CAR/TCR transgene to increase the in vivo persistence of the engineered T-cells for increased efficacy (Majzner and Mackall, 2019, Nat. Med.). The instant methods provide improved safety characteristics. By limiting the CAR/TCR expression to a few days in vivo, the autologous, engineered T-cells will revert to their native specificity, which may reduce or prevent treatment-related adverse events. At the same time, the transient expression of genes encoding the proteins described herein produce T cells that are resistant to the immunosuppressive effect for only a limited temporal window (about 72 hours). The ability to engineer T-cells with such transient expression characteristics provides an advance in the field and solves a problem that is not addressed by current methods.

Second, it was not predictable that the transient expression of the mutant immunosuppressant inhibitors through mRNA electroporation would have the desired immunosuppressant resistance effect. In prior studies where T-cells were made to be resistant to tacrolimus or MMF (Brewin et al, 2009, Blood) (Jonnalagadda et al, 2013, Plos One), the expression of the mutated forms of CnB and IMPDH was constitutive and mediated by the use of viral vector transduction. The consistent source of the mutant proteins could clearly out-compete the wild-type protein and hence confer the resistance to tacrolimus and MMF. Therefore, when the expression of immunosuppressant inhibitors occurred only for a short duration, it was not predictable whether the quantities of mutant protein generated would be sufficient to out-compete the wild-type protein.

Third, the methods described herein concurrently electroporate the mRNA encoding the antigen-specific TCR, and mRNA encoding one or more immunosuppressant inhibitors. In some embodiments, the methods described herein concurrently electroporate the mRNA encoding the HBV-specific TCR, mutant CnB and mutant IMPDH into a T cell, where the mRNA exist as 3 independent mRNA constructs. Similar to above, the effectiveness of simultaneously electroporating 3 independent mRNA constructs cannot be predicted without experimentation.

Taken together, the instant methods and compositions provide the unexpected advantages of i) transient expression of the mutant immunosuppressant inhibitor proteins through mRNA electroporation, and ii) the ability to electroporate multiple mRNA constructs into a single cell. Furthermore, the general focus of the field is on viral vector transduction methods rather than electroporation of multiple, independent mRNA constructs as in the instant methods.

### Modified T Cells

Described herein are modified T cells that express both an exogenous TCR and an exogenous immunosuppressant inhibitor. The modified T cells can be transfected (e.g., electroporated) or transduced with mRNA encoding the exogenous TCR and mRNA encoding the exogenous immunosuppressant inhibitor.

In some embodiments, the exogenous TCR specifically binds an antigen expressed by a cell. In some embodiments, the antigen is expressed by a tumor cell. In some embodiments, the antigen is a viral antigen. In some embodiments, the exogenous TCR specifically binds an epitope from a virus, such as hepatitis B virus (HBV), cytomegalovirus (CMV) or Epstein-Barr virus (EBV).

In some embodiments, the TCR comprises alpha and beta chains that specifically bind the s183-191 peptide of HCV (referred to herein as s183-TCR). In some embodiments, the TCR specifically binds a HBV core antigen comprising amino acids 18-27 of the intact protein. In some embodiments, the TCR binds an epitope from the LMP2 protein of EBV.

In some embodiments, the viral antigen is expressed by a tumor cell. In some embodiments, the tumor cell is from a liver tumor. In some embodiments, the tumor cell is from a hepatocellular carcinoma (HCC) tumor. In some embodiments, the tumor cell comprises a viral DNA inserted into the cell's genome. For example, in some embodiments, HCC tumor cells comprise HBV-DNA integrated into the cell's genome. In some embodiments, the viral DNA is an etiologic agent that is associated with or causative of the transformed or neoplastic tumor cell phenotype.

In some embodiments, the antigen is expressed by a cell infected with a virus. In some embodiments, the virally infected cell is present in an immunosuppressed subject or patient. In some embodiments, the virus is CMV or EBV.

In some embodiments, the modified T cells comprise mRNA encoding an exogenous TCR. In some embodiments, the alpha and beta chains of the TCR are translated from a single mRNA molecule comprising nucleic acid sequences encoding the alpha and beta chains of the TCR. In some embodiments, the modified T cells comprise mRNA encoding an exogenous TCR that binds to antigens or epitopes expressed by a tumor cell. In some embodiments, the modified T cells comprise mRNA encoding an exogenous TCR that binds to antigens or epitopes derived from a virus, such as HBV, CMV, or EBV.

In some embodiments, the exogenous TCRs described herein are functional in the modified T cell in which they are expressed. In particular, the exogenous TCRs may be functional heterodimers of alpha and beta TCR chains associated with a CD3 complex that recognize at least one epitope in the context of at least one Class I or Class II MHC molecule. In humans, the MEC restriction of at least one epitope may be dependent on at least one particular Human Leukocyte Antigen (HLA) expressed by at least one cell presenting the antigen. TCRs that bind viral epitopes can be restricted to any HLA type (i.e., HLA-A, HLA-B, HLA-C, HLA-DPA1, HLA-DPB1, HLA-DQA1, HLA-DQB1, HLA-DRA, and HLA-DRB1). In some embodiments, the exogenous TCR may recognize at least one epitope in the context of at least one MEC molecule of at least one species other than human, e.g., H-2K of mouse.

In some embodiments, the TCR recognizes and binds to HBV epitopes that are HLA-A2 restricted. Approximately 50% of the general population express the MHC class I molecule HLA-A2, an HLA-A serotype. Therefore, HLA-A2-restricted TCRs may find widespread therapeutic use. In particular, the subtype may identify gene products of many HLA-A*02 alleles, comprising HLA-A*0201, *0202, *0203, *0206, and *0207 gene products. There may be distinct differences in the subtypes between Caucasian and Asian populations. Whereas more than 95% of the HLA-A2 positive Caucasian population is HLA-A0201, the HLA-A2 positive Chinese population may be broken down into 23% HLA-A0201; 45% HLA-A0207; 8% HLA-A0206; 23% HLA-A0203.

The TCRs described herein may be HBV-epitope reactive. A list of known immunoreactive HBV epitopes and their sequences may be found in “Immunodominance: The choice of the Immune System,” J. A. Frelinger, ed (Weinheim: Wiley-VCH) (see page 233, chapter 11 The effect of pathogens on the immune system: Viral hepatitis), which is herein incorporated by reference. The HBV epitope may comprise at least one core antigen, envelope antigen, surface antigen and/or mutants thereof.

In some embodiments, the TCR specifically binds a viral epitope from an HBV, EBV, CMV, FLU or SARS virus. In some embodiments, the TCR specifically binds a viral epitope listed in Table 1 below (see, Banu et al., Building and Optimizing a Virus-specific T Cell Receptor Library for Targeted Immunotherapy in Viral Infections, Sci Rep. 2015; 4: 4166):

TABLE 1
Cloned Virus-specific T cell receptors

| | | | aa | Peptide | | Optimal | | | IFN- |
|---|---|---|---|---|---|---|---|---|---|
| # | Virus | Ag | position | Sequence | HLA | orientation | Vβ^{a} | Pent^{a} | γ^{a} |
| 1 | CMV | IE1 | 42-50 | KEVNSQLSL | B4001 | Vβ27-P2A- | 1.2 | 3.5 | 1.3 |
| | | | | | | Vα26 | | | |
| 2 | CMV | pp65 | 501-09 | ATVQGQNLK | A1101 | Vβ9-P2A- | 4.1 | 8.1 | 1.6 |
| | | | | | | Vα29 | | | |
| 3 | CMV | pp65 | 495-505 | NLVPMVATV | A0201 | Vβ12-P2A- | 1.1 | 1 | 1.2 |
| | | | | | | Vα5 | | | |
| 4 | EBV | EBNA- | 399-408 | AVFDRKSDAK | A11 | Vβ5-P2A- | 2.4 | | 3.2 |
| | | 4NP | | | | Vα19 | | | |
| 5 | HBV | env | 171-80 | FLGPLLVLQA | Cw0801 | Vβ20.1- | 2.5 | 16.9 | 6.3 |
| | | | | | | P2A-Vα5 | | | |
| 6 | HBV | core | 18-27 | FLPSDFFPSV | A0201 | Vα17-P2A- | 2.9 | 2.4 | 3.7 |
| | | | | | | Vβ12-4 | | | |
| 7 | HBV | env | 370-379 | SIVSPFIPLL | A0201 | Vβ7.8-P2A- | | 9.8 | 5.4 |
| | | | | | | Vα12 | | | |
| 8 | HBV | env | 183-191 | FLLTRILTI | A0201 | Vβ28-P2A- | 1.2 | 1.9 | 1.9 |
| | | | | | | Vα34.1 | | | |
| 9 | SARS | NP | 216-225 | GETALALLLL | B4001 | Vβ4.3-P2A- | 1 | 2.7 | 1.4 |
| | | | | | | Vα4.1 | | | |
| 10 | Flu | M1 | 58-66 | GILGFVFTL | A0201 | Vβ19.1- | 1 | 1.3 | 1.1 |
| | | | | | | P2A-Vα27 | | | |
| | | | | | | Mean | 1.9 | 4.2 | 2.7 |

^{a}Fold increase based on positive orientation of TCR cassette.

Antigen-specific T cells can be identified using matching HLA-pentamers/tetramers or the CD107a degranulation assay and clonal populations can be derived by limiting dilution cloning or sorting T cells using antibodies specific for the variable region of TCR beta chains. The TCRs can be cloned by extracting total RNA from sorted clones and the wild type TCR alpha and beta genes cloned using rapid amplification of cDNA ends (RACE) PCR with TCR constant region gene specific primers. The TCRs can be cloned into a suitable vector, such as a retroviral vector, and tested for expression in primary human T cells.

In some embodiments, the modified T cells comprise mRNA encoding an exogenous polypeptide or protein inhibitor of an immunosuppressant. In some embodiments, the immunosuppressant is Tacrolimus or mycophenolate mofetil (MMF). In some embodiments, the mRNA encodes an exogenous polypeptide or protein that reduces expression of the FK506-binding protein (FKBP1A). In some embodiments, the mRNA encodes a mutant calcineurin (CN) subunit B (CnB) protein. In some embodiments, the mRNA encodes an exogenous polypeptide or protein that blocks or reduces MPA binding to IMPDH. In some embodiments, the mRNA encodes a mutant IMPDH protein.

In some embodiments, the modified T cell comprises exogenous nucleic acids that reduce or inhibit expression of endogenous mRNA expressed by a tumor cell. In some embodiments, the exogenous nucleic acids comprise small-interfering RNAs (si-RNA) or micro RNA (miRNA).

In some embodiments, the modified T cell is an activated T cell. In some embodiments, the activated T cells are isolated from peripheral blood mononuclear cells (PBMC) of a subject. Activated T cells can be isolated using methods know in the art, including flow cytometry and Fluorescent Activated Cell Sorting (FACS) analysis. Activated T cells from humans can be identified by expression of one more markers selected from CD8, CD39, or HLA-DR, or by the production of cytokines after antigen specific stimulation. In some embodiments, the T cell expresses CD4. In some embodiments, the modified T cells transiently express both the native (endogenous) and exogenous TCR. In some embodiments, the native (endogenous) TCR is not determined, but knowledge of the endogenous TCR is not necessarily required for the methods described herein.

### Inhibitors of Immunosuppressants

Described herein are agents that confer resistance to an immunosuppressant, or reduce the activity of an immunosuppressant. In some embodiments, the agent comprises a molecule or compound that decreases expression of a gene or protein in an immunosuppressant pathway. In some embodiments, the agent comprises a nucleic acid that inhibits expression of an mRNA encoding a gene or protein in an immunosuppressant pathway. In some embodiments, the nucleic acid is an interfering RNA, such as siRNA. In some embodiments, the agent comprises a mutated version of the gene or protein. In some embodiments, the agent is overexpressed in the modified cell.

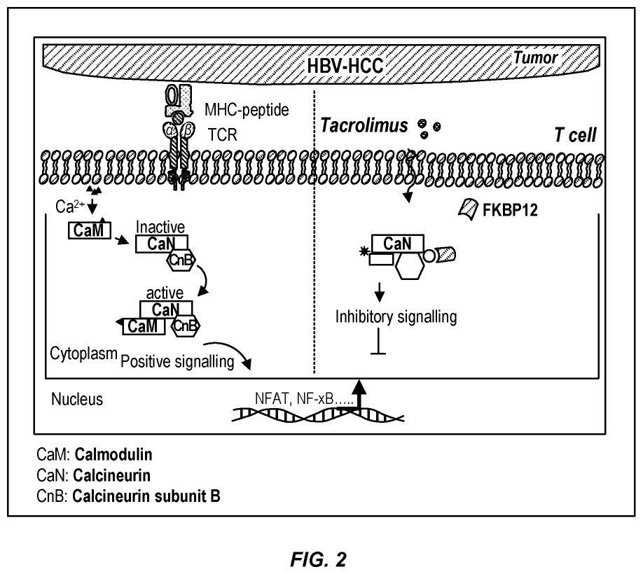

In some embodiments, the immunosuppressant is Tacrolimus (FK506), and the inhibitor of Tacrolimus is a nucleic acid or protein that reduces expression of the FK506-binding protein (FKBP1A). As shown in FIG. 2, the FK506-FKBP1A complex binds to the calcineurin (CN) heterodimer, which subsequently blocks NFAT pathway activation. Thus, in some embodiments, the immunosuppressant pathway is a pathway that normally activates the NFAT/NF-kappaB pathway or the calcineurin pathway. In some embodiments, the inhibitor of Tacrolimus is a nucleic acid, such as an si-RNA, that inhibits expression of FKBP1A. In some embodiments, the inhibitor of Tacrolimus is a mutant calcineurin (CN) subunit B (CnB) protein.

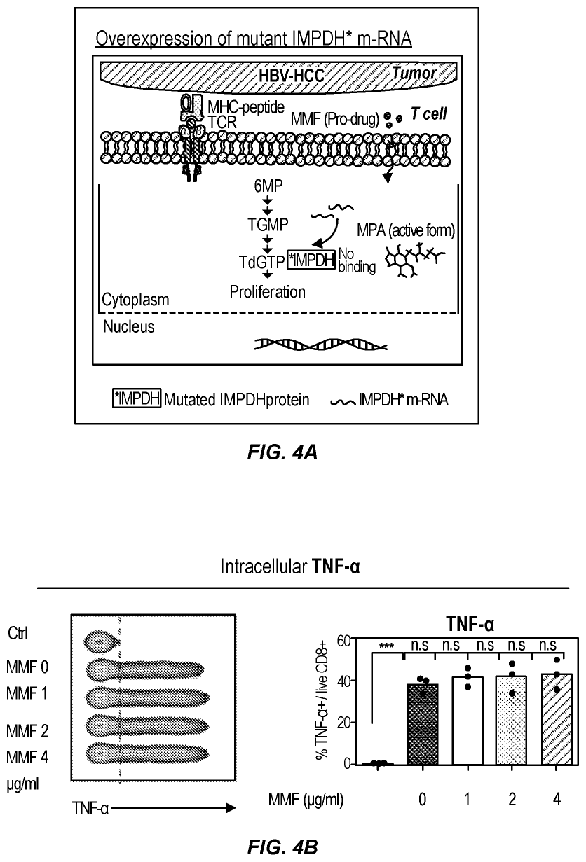

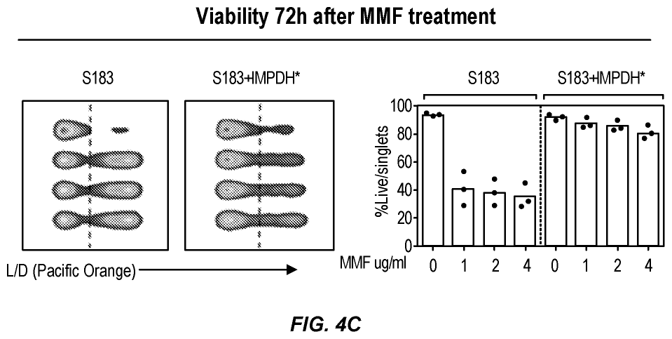

In some embodiments, the immunosuppressant is Mycophenolate mofetil (MMF). MMF is an anti-metabolite drug used as an adjunctive immunosuppressive agent in combination with tacrolimus. MMF reduces the cytotoxic effect of tacrolimus particularly in patients with renal dysfunction and neurotoxicity. MMF is a pro-drug that rapidly hydrolyses to its active form, MPA, within the liver. MPA inhibits inosine 5′-monophosphate dehydrogenase (IMPDH) which is essential for de novo purine synthesis and selectively inhibits lymphocyte proliferation (FIG. 4A). Typically, a standard fixed dose of 1-2 g MMF is given twice a day to achieve maintenance immunosuppression (serum trough level—1-3 pg/ml). In some embodiments, the inhibitor of MMF is an agent that blocks or reduces binding of MPA to IMPDH. As shown in FIG. 4, overexpression of mutant IMPDH recovers T cells viability in the presence of therapeutic concentrations of MMF. Thus, in some embodiments, the inhibitor of MMF comprises a mutant IMPDH protein.

In some embodiments, the mutant CnB and IMPDH sequences are codon optimized for expression in T cells.

### Methods for Producing Modified T Cells

Also described are methods for producing the modified T cells described herein. In some embodiments, the methods comprise introducing exogenous nucleic acids into T cells. Constructs comprising exogenous nucleic acids can be delivered to cells in vitro, ex vivo or in vivo using any number of methods known to those of skill in the art. For example, if the cells are in vitro or ex vivo, they can be transformed or transduced according to standard protocols, e.g., those described in Molecular Cloning: A Laboratory Manual (Fourth Edition), by M. R. Green and J. Sambrook, (2012). Examples of suitable methods include but are not limited to, the CaCl₂) chemical method or electroporation. In some embodiments, the exogenous nucleic acids are introduced into T cells by electroporation. In some embodiments, one or more independent mRNAs are introduced into one or more T cells. In some embodiments, one, two, three or more independent mRNAs are introduced into one or more T cells. In some embodiments, the exogenous nucleic acids are introduced into T cells in vivo, for example by viral vectors, nanoparticles, gold particles, lipoplexes and/or polyplexes.

In some embodiments, the modified T cell is an autologous T cell isolated from a subject. In some embodiments, the modified T cell is an autologous T cell isolated from a subject having a disease. In some embodiments, the modified T cell is an autologous T cell isolated from a subject having liver cancer. In some embodiments, the modified T cell is an autologous T cell isolated from a subject having HCC.

In some embodiments, a construct comprising a polynucleotide encoding an exogenous TCR or immunosuppressant inhibitor described herein can be inserted or cloned into a suitable expression vector. In some embodiments, a construct comprising a polynucleotide encoding an exogenous TCR or immunosuppressant inhibitor described herein is operably connected to at least one promoter. The coding sequences for alpha and beta-chains of the TCR can be operably connected to at least one promoter functional in the isolated T cell. Suitable promoters may be constitutive and inducible promoters, and the selection of an appropriate promoter is well within the skill in the art. For example, suitable promoters may comprise, but are not limited to, the retroviral LTR, the SV40 promoter, the CMV promoter and cellular promoters (e.g., the beta-actin promoter).

According to one aspect, the present invention provides at least one method of preparing at least one T cell comprising at least one HBV epitope-reactive exogenous TCR for delivery to at least one subject comprising transducing at least one T cell isolated from the subject with the construct of and/or the vector of the present invention. Constructs and vectors according to the present invention may be delivered to cells in vitro, ex vivo or in vivo using any number of methods known to those of skill in the art. For example, if the cells are in vitro or ex vivo, they may be transformed or transduced according to standard protocols, e.g., those described in Molecular Cloning: A Laboratory Manual, 3d ed., Sambrook and Russell, CSHL Press (2001), incorporated herein by reference. Examples of methods may comprise but are not limited to, the CaCl₂) chemical method, electroporation and the like. In particular, the constructs according to the present invention may be delivered into the cells in vivo. Suitable methods of delivery of polynucleotide constructs are known in the art, and may comprise but are not limited to, viral vectors, nanoparticles, gold particles, lipoplexes and/or polyplexes.

### Methods of Treatment and Diagnosis

Also provided are methods of treating a medical condition or disease in a subject or patient by administering the modified immunosuppressant resistant T cells described herein to the subject or patient. Methods of treatment can reduce the number or severity of symptoms associated with a medical condition or disease, or can prevent or completely eliminate (cure) the medical condition or disease. The subject or patient can be an animal, a mammal, or a human. In some embodiments, a modified T cell described herein is administered to a subject in need of treatment. In some embodiments, the medical condition or disease is cancer, a tumor, or a viral infection. In some embodiments, the disease is HCC. In some embodiments, the treatment may be used to cure or prevent an acute or chronic HBV infection or an associated condition, including hepatocellular carcinoma.

In some embodiments, viral infections can be treated by administering the immunosuppressant resistant T-cells described herein. In some embodiments, the viral infections are HBV, CMV and EBV infections. CMV and EBV infect almost all adults globally and while these viruses remain latent and do not cause overt pathologies under normal circumstances, immunosuppression of patients with organ or stem cell transplantation often cause a reactivation of these viruses, which can lead to the respective virus-related disorders or graft rejection and consequently increased mortality. Thus, in some embodiments, modified immunosuppressant resistant T cells that express TCRs that bind to antigens or epitopes from HBV, CMV or EBV are administered to a subject in need of treatment.

In some embodiments, a therapeutically effective amount of the modified T cells described herein is administered to the subject. As will be understood by the skilled person, the quantity of cells that make up the immunotherapeutically effective amount of cells to be administered depends on the subject to be treated. This may be dependent on but not limited to, the capacity of the individual's immune system to mount TCR-mediated immune response, the age, sex and weight of the patient and the severity of the condition being treated. The number of variables in regard to at least one individual's prophylactic or treatment regimen may be large, and a considerable range of doses may be expected. In particular, cells may be administered in at least one amount from 5×10⁵ cells/kg body weight to 1×10¹⁰ cells/kg body weight, for example, 5×10⁶ cells/kg body weight to 1×10⁸ cells/kg body weight may be administered. The maximal dosage of cells to be administered to the subject may be the highest dosage that does not cause undesirable and/or intolerable side effects. Suitable regimens for initial administration and additional treatments may also be contemplated and may be determined according to conventional protocols.

Also provided are modified T cells described herein for use in the treatment of a tumor or a viral infection. In some embodiments, the modified T cells described herein are for use in the treatment of a HBV infection and/or HBV-related hepatocellular carcinoma.

According to another aspect, also provided is a modified immunosuppressant resistant T cell described herein for the preparation of a medicament for treating a tumor or viral infection.

Suitable solid or liquid medicament preparation forms may be, for example, granules, powders, tablets, coated tablets, (micro) capsules, suppositories, syrups, emulsions, suspensions, creams, aerosols, drops or injectable solutions in ampule form and also preparations with protracted release of active compounds, in whose preparation excipients and additives and/or auxiliaries such as disintegrants, binders, coating agents, swelling agents, lubricants, flavourings, sweeteners or solubilizers are customarily used as described above. The medicaments may be suitable for use in a variety of drug delivery systems.

### EXAMPLES

### Example 1

An exemplary method for producing the modified immunosuppressant resistant T cells described herein.

PBMC of healthy subjects were cultured with 50 ng/ml anti-CD3 and 600 IU/ml IL-2 in T cell media containing AIM-V 2% human AB serum for 7 days. On day 7, IL-2 concentration was increased to 1000 IU/ml and the T cells were incubated overnight. On day 8, expanded/activated T cells were electroporated with 3 mRNAs. In brief, 10×10⁶ activated T cells were washed 3 times with electroporation media. 20 μg of S183 mRNA, 20 μg of mutant IMPDH mRNA and 10 μg of mutant CnB mRNA were added to T cells followed by addition of 200 μl of electroporation media. The mixture was transferred to a 4 mm cuvette and electroporated via customized program of Agile Pulse electroporation system (Harvard Bioscience). Electroporated T cells were rested for 2 minutes and maintained overnight in AIM-V media containing 10% human AB serum plus 100 IU/ml rIL-2 at 37° C. and 5% CO₂. TCR expression was quantified 24 hours post-electroporation.

### Example 2

Engineered T cells that express an exogenous TCR and an inhibitor of Tacrolimus.

As shown in FIG. 2, Tacrolimus diffuses into the T cell cytoplasm and binds to its 12-kDa chaperone protein called FK506-binding protein (FKBP1A). This small complex binds to the CN heterodimer, which subsequently blocks NFAT pathway activation.

As a first strategy, smart pool si-RNA was used to knockdown tacrolimus binding protein FKBP1A. Concurrent electroporation of si-RNA and engineered TCR mRNA can partially recover T cell polyfunctionality and cytolytic activity.

To improve the response, the CnB binding site in the CN complex was targeted. Based on previous evidence, mutation in CnB inhibits docking of either or both FK506/FKBP12 and CsA/CyPA complexes, but does not affect NFAT dephosphorylation. Previous studies using viral transduction of mutant CnB30 in EBV-specific T cells showed resistance to both Tacrolimus and cyclosporine A (CsA) (4). Hence, mutant CnB30 mRNA was in vitro transcribed and electroporated into T cells concurrently with the mRNA coding for the HBV TCR.

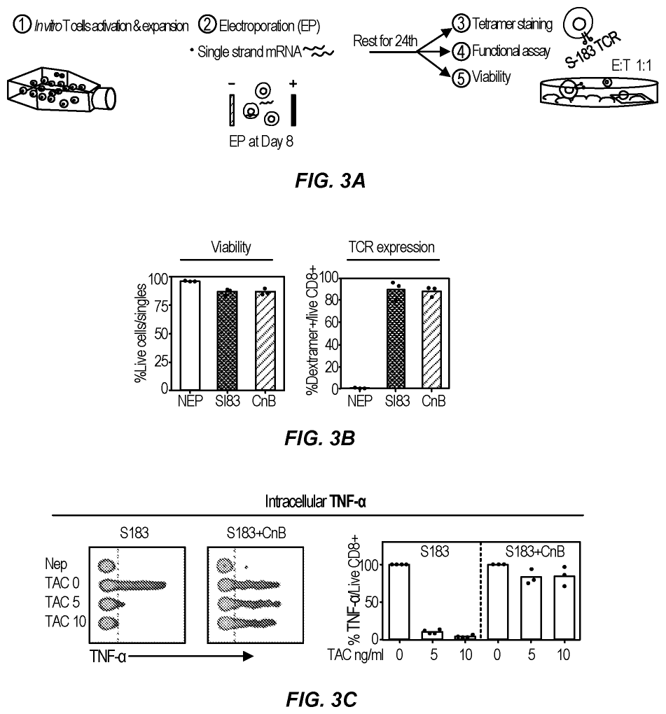

Results: Concurrent electroporation of S-183 TCR and mutant CnB showed profound functional recovery (approximately 90%) (FIG. 3C) without any interference in S183 TCR expression. Notably, this expression only lasted for about 72 hours post-electroporation, after which the T cells regained their sensitivity to tacrolimus. The transient expression of the mRNAs improves safety and reduces the potential risk of liver graft rejection when used in HBV-HCC patients who have received liver transplantation.

This example demonstrates that concurrent overexpression of CnB mutant protein, through mRNA electroporation, in HBV-TCR engineered T-cells results in T cells that are transiently resistant to Tacrolimus, and have improved functional activity compared to T cells that express HBV-TCR alone.

### Example 3

Engineered T cells that express an exogenous TCR and an inhibitor of Mycophenolate mofetil (MMF).

Mycophenolate mofetil (MMF) is a common immunosuppressant frequently used as an adjunctive immunosuppressive agent in combination with tacrolimus. It reduces the cytotoxic effect of tacrolimus, particularly in patients with renal dysfunction and neurotoxicity. MMF is a pro-drug that rapidly hydrolyses to its active form, MPA, within the liver. MPA inhibits inosine 5′-monophosphate dehydrogenase (IMPDH), which is essential for de novo purine synthesis and selectively inhibits lymphocyte proliferation (FIG. 4A). Typically, a standard fixed dose of 1-2 g MMF has been given twice a day to achieve maintenance immunosuppression (serum trough level approximately 1-3 pg/ml).

According to the data shown in FIGS. 4B and 4C, a clinically relevant concentration of MMF does not impair T cell function and killing, but markedly decreases the viability of the modified TCR-T cells' viability after 48 hours exposure to the drug (see FIG. 4C. “Mock EP”). Therefore, T cells were concurrently electroporated with mRNA encoding the HBV-TCR (s183) and mRNA encoding a mutant IMPDH.

Results: T cells engineered to express both HBV-TCR and a mutated IMPDH showed dramatic improvement in cell viability (FIG. 4C, “IMPDH*”).

This example demonstrates that using mRNA electroporation, the engineered HBV-TCR T cells that express mutated IMPDH can markedly maintain their viability for up to 72 hours in the presence of MMF.

### Example 4

A representative method of treating a patient with HCC.

An adoptive T-cell immunotherapy strategy has been developed, wherein autologous T-cells isolated from the peripheral blood of HCC patients were engineered to be specific for HBV peptides presented on the surface of the HCC cells (HBV-TCR T-cells). As a proof-of-concept, two patients with HBV-HCC relapses after liver transplantation have been treated with the modified T cells in a compassionate setting, and in one patient, a prominent anti-tumour response was observed, followed by a stabilization of disease progression for almost two years. However, such patients will also need to be administered life-long immunosuppressant to prevent rejection of the liver graft. The present disclosure provides an alternative method for treating patients with HCC.

Recent in vitro data showed that the immunosuppressants can profoundly inhibit the function of engineered HBV-TCR T-cells. As such, we determined if a clinically used immunosuppressant could interfere with the function of the HBV-TCR T-cells, and whether immunosuppressant resistant HBV-TCR T-cells could be engineered.

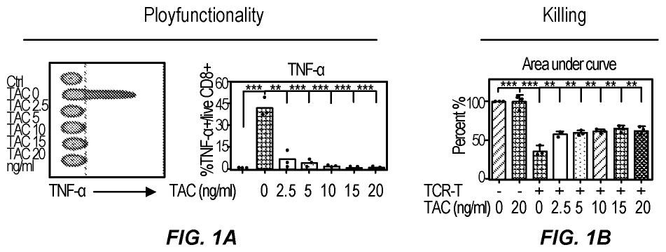

First, the function of HBV-TCR T-cells in the presence of a widely used immunosuppressant, Tacrolimus, at clinically relevant doses was assessed. The presence of Tacrolimus can potently inhibit both the cytotoxic function and cytokine secretion of HBV-TCR T-cells. As shown in FIG. 1A, a clinically relevant concentration of Tacrolimus can impair T cell TNF-α production following 12 hours incubation with the HBV-antigen-expressing target cells (i.e. HepG2.2.15 cells). Tacrolimus-treated T cells also lost their cytolytic activity up to a maximum of 50% of the non-treated control (FIG. 1B).

To overcome the negative effects of Tacrolimus on the engineered T cells, autologous T cells that have been modified to express both an exogenous TCR and an exogenous immunosuppressant inhibitor are tested in vitro and in vivo. In vitro assays described above are used to determine the cytotoxic function and cytokine secretion of HBV-TCR T-cells. The modified T cells are tested in an in vivo model of HCC, such as those described in Heindryckx F, Colle I, Van Vlierberghe H. Experimental mouse models for hepatocellular carcinoma research. Int J Exp Pathol. 2009; 90(4):367-386. Examples of suitable models include xenograft models, which develop HCC by implanting hepatoma cell lines in mice, either ectopically or orthotopically. A suitable animal model is described in Koh, S. et al., “A Practical Approach to Immunotherapy of Hepatocellular Carcinoma Using T Cells Redirected Against Hepatitis B Virus,” Mol Ther Nucleic Acids. 2013; 2(8):e114.

The autologous T cells that are tested for efficacy can be modified to express both a HBV-specific TCR and a mutant protein. The modified autologous T cells that show efficacy in vitro and/or in vivo are then administered to a subject to determine if they produce an anti-tumor response.

### Example 5

Reducing the Expression of FKBP1A in T Cells Provides Resistance to Tacrolimus

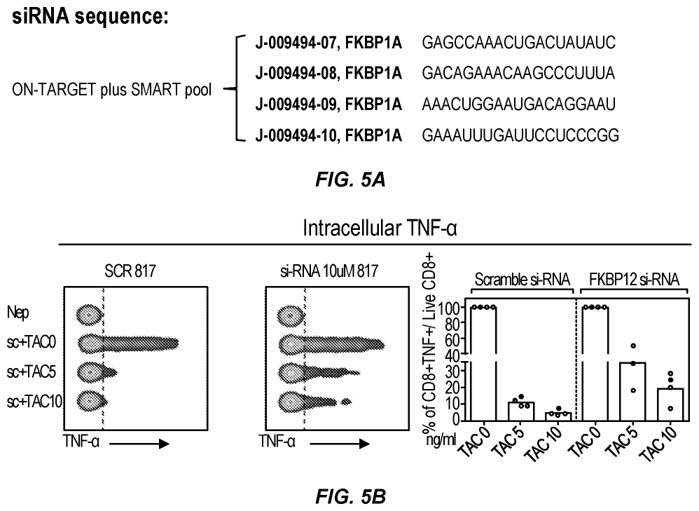

An si-RNA electroporation system was adopted to make HBV-TCR T cells transiently resistant to tacrolimus. In the si-RNA approach, FKBP12 ON-TARGET plus si-RNA and m-RNA encoding HBV envelope s183-TCR were concomitantly delivered to the T cells, using nucleofection. With this strategy, FKBP1A m-RNA expression was knocked down by close to 100% 24 hours after electroporation, with no impairment to T cell viability or HBV-TCR kinetics. As shown in FIG. 5B, siRNA-mediated knockdown of FKBP1A can only partially recover (˜20% recovery with 5 ng/ml of Tacrolimus; 10% Scramble VS 30% FKBP12) T cell function in the presence of Tacrolimus. This partial recovery is perhaps explained by the long half-life of previous FKBP1A protein inside the cells, which may have reduced the si-RNA-mediated effect. Comparing these findings with the data obtained through overexpression of mutant CnB, where functional recovery of HBV-TCR T cells in the presence of Tacrolimus is ˜90% (FIG. 3C), shows that the latter approach has significant advantages over FKBP1A siRNA knockdown for developing Tacrolimus-resistant TCR-T cells.

### Example 6

T cells concurrently electroporated with mRNAs encoding an HBV-specific TCR, a mutant CnB and a mutant IMPDH are resistant to both TAC and MMF.

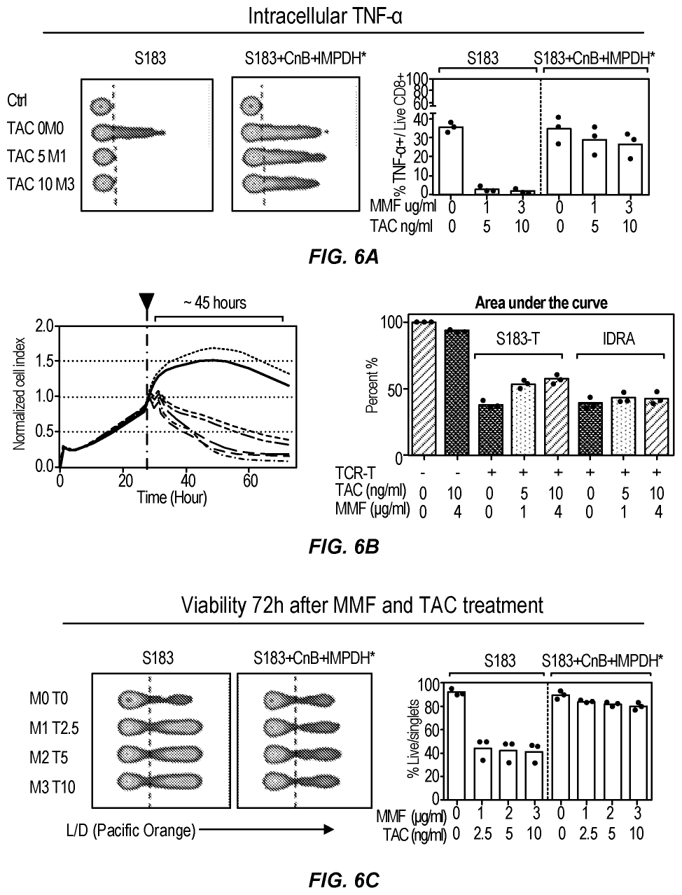

To check the possibility that dual resistant T cell for MMF and Tacrolimus could be developed, all 3 m-RNA including mutant CnB, mutant IMPDH and env-183 TCR were concomitantly electroporated to the T cells, and their function and viability evaluated in the presence and absence of drugs. Concomitant electroporation of s183-TCR, CnB and IMPDH had only a minor impact on TCR expression (˜10-20% reduction) and viability (up to 15%) of engineered T cells 24 hours post-electroporation. Cytokine analysis of these T cells showed that 3 m-RNA electroporated T cells can retain their Ag-specific function in the presence of both immunosuppressants (FIG. 6A). This feature remains transiently, and engineered T cells subsequently become sensitive to drugs after 5 days. As described earlier, MMF's major effect is on the viability of T cells. Viability analysis of cells after a 72-hour exposure with both drugs showed that dual-resistant T cells retain viability almost the same as non-treated T cells (FIG. 6B). Herein is described, for the first time, dual-resistant T cell that can be used for cell therapy applications in obligate immunosuppression.

### Example 7

T cells concurrently electroporated with mRNAs encoding an EBV-specific TCR, a mutant CnB and a mutant IMPDH are resistant to both TAC and MMF.

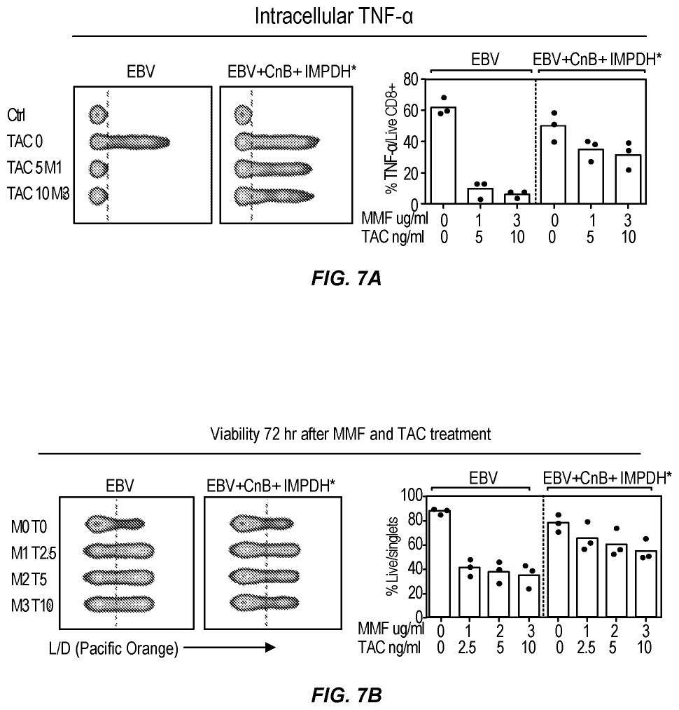

To demonstrate the broad applicability of the present invention in the context of T cell therapies in obligate immunosuppression, IDRA EBV-redirected T cells were genetically developed. As expected, in the presence of clinically relevant concentration of tacrolimus and MMF, EBV redirected T cells lost their polyfunctionality and viability (FIGS. 7A and 7B). Concomitant electroporation of EBV TCR m-RNA together with mutant CnB and IMPDH could dramatically recover the function and viability of these T cells in the presence of immunosuppressants agents (FIGS. 7A and 7B). These findings show that the approach of the present invention may be successfully applied in other cell therapy situations where the use of immunosuppression is indispensable.

All patents, patent applications, and other publications, including GenBank Accession Numbers, cited in this application are incorporated by reference in the entirety for all purposes.

### REFERENCES

1. Savoldo B, Goss J, Liu Z, Huls M H, Doster S, Gee A P, Brenner M K, Heston H E, Rooney C M. Generation of autologous Epstein-Barr virus-specific cytotoxic T cells for adoptive immunotherapy in solid organ transplant recipients. Transplantation. 2001 Sep. 27; 72(6):1078-86. PubMed PMID: 11579304.
2. Zhan X, Brown B, Slobod K S, Hurwitz J L. Inhibition of ex vivo-expanded cytotoxic T-lymphocyte function by high-dose cyclosporine. Transplantation. 2003 Aug. 27; 76(4):739-40. PubMed PMID: 12973121.
3. Laskin B L, Jiao J, Balualte H J, Amaral S, Furth S L, Akimova T, Hancock W W, Levine M H, Reese P P, Beier U H. The Effects of Tacrolimus on T-Cell Proliferation Are Short-Lived: A Pilot Analysis of Immune Function Testing. Transplant Direct 2017 Jul. 19; 3(8): e199. doi: 10.1097/7XD.0000000000000715. eCollection 2017 August PubMed PMID: 28795150; PubMed Central PMCID: PMC5540637.
4. Brewin J, Mancao C, Straathof K, Karlsson H, Samarasinghe S, Amrolia P J, Pule M. Generation of EBV-specific cytotoxic T cells that are resistant to calcineurininhibitors for the treatment of post transplantation lymphoproliferative disease. Blood. 2009 Nov. 26; 114(23):4792-803. doi: 10.1182/blood-2009-07-228387. Epub 2009 Sep. 21. PubMed PMID: 19770360.
5. Jonnalagadda M, Brown C E, Chang W C, Ostberg J R, Forman S J, Jensen M C. Engineering human T cells for resistance to methotrexate and mycophenolate mofetil as an in vivo cell selection strategy. PLoS One. 2013 Jun. 6; 8(6):e65519. doi: 10.1371/joumatpone.0065519. PubMed PMID: 23755242
6. Majzner R G, Mackall C L. Clinical lessons learned from the first leg of the CAR T cell journey. Nat Med. 2019 September; 25(9):1341-1355.
7. Koh, S. et al., A practical approach to immunotherapy of hepatocellular carcinoma using T cells redirected against hepatitis B virus. Mol Ther Nucleic Acids. 2013 Aug. 13; 2:e 114.
8. U.S. Pat. No. 8,603,810, Bertoletti et al., Agency for Science, Technology and Research, Singapore (SG).
9. Tan A T, Yang N, Lee Krishnamoorthy T, et al. Use of Expression Profiles of HBV-DNA Integrated Into Genomes of Hepatocellular Carcinoma Cells to Select T Cells for Immunotherapy. Gastroenterology. 2019; 156(6):1862-1876.el 869. doi:10.1053/j.gastro.2019.01.251.
10. Banu N, Chia A, Ho Z Z, et al. Building and optimizing a virus-specific T cell receptor library for targeted immunotherapy in viral infections. Sci Rep. 2014; 4:4166. doi:10.1038/srep04166.
11. Koh S, Shimasaki N, Bertoletti A. Redirecting T Cell Specificity Using T Cell Receptor Messenger RNA Electroporation. Methods Mol Biol. 2016; 1428(Chapter 19):285-296. doi: 10.1007/978-1-4939-3625-0_19.

INFORMAL SEQUENCE LISTING:
CnB30 coding region sequence (SEQ ID NO: 1):
ATGGGCAACGAGGCCAGCTACCCTCTGGAGATGTGCTCCCACTTCGACGCCGACGA
GATCAAGCGGCTGGGCAAGCGCTTCAAGAAGCTGGACCTGGACAACAGCGGCAGCC
TGAGCGTGGAGGAGTTTATGTCTCTGCCCGAGCTGCAGCAGAACCCCCTGGTGCAGC
GCGTGATCGACATCTTCGACACCGACGGCAACGGCGAGGTGGACTTCAAGGAGTTC
ATCGAGGGCGTGAGCCAGTTCAGCGTGAAGGGCGACAAGGAGCAGAAGCTGCGGTT
CGCCTTCCGGATCTACGATATGGATAAAGATGGCTATATTTCTAATGGCGAGCTGTT
CCAGGTGCTGAAGATGATGGTGGGCAACAATACCAAGCTGGCCGATACCCAGCTGC
AGCAGATCGTGGACAAGACCATCATCAACGCCGACAAGGACGGCGACGGCAGAAT
CAGCTTCGAGGAGTTCTGTGCCGTGGTGGGAGGCCTGGATATTCACAAAAAAATGG
TGGTGGACGTGTGA
CnB30 coding region sequence, codon optimized (SEQ ID NO: 2):
ATGGGGAATGAGGCATCCTATCCACTGGAAATGTGCAGCCACTTCGACGCCGACGA
AATCAAAAGACTGGGGAAAAGGTTCAAAAAGCTGGACCTGGATAACAGCGGCTCCC
TGTCTGTGGAGGAGTTCATGTCCCTGCCCGAGCTGCAGCAGAACCCTCTGGTGCAGA
GAGTGATCGACATCTTTGACACCGATGGCAATGGCGAGGTGGATTTCAAGGAGTTTA
TCGAGGGCGTGAGCCAGTTCTCCGTGAAGGGCGACAAGGAGCAGAAGCTGCGGTTC
GCCTTTAGAATCTACGACATGGATAAGGACGGCTATATCTCTAACGGCGAGCTGTTT
CAGGTGCTGAAGATGATGGTGGGCAACAATACCAAGCTGGCCGACACACAGCTGCA
GCAGATCGTGGATAAGACAATCATCAATGCCGATAAGGACGGCGATGGCCGGATCA
GCTTCGAGGAGTTCTGTGCCGTGGTCGGAGGGCTGGATATTCATAAAAAGATGGTCG
TCGATGTCTGA
M-IMPDH2^{R}; T333I, S351Y CDS (SEQ ID NO: 3):
ATGGCCGACTACCTGATTAGTGGGGGCACGTCCTACGTGCCAGACGACGGACTCAC
AGCACAGCAGCTCTTCAACTGCGGAGACGGCCTCACCTACAATGACTTTCTCATTCT
CCCTGGGTACATCGACTTCACTGCAGACCAGGTGGACCTGACTTCTGCTCTGACCAA
GAAAATCACTCTTAAGACCCCACTGGTTTCCTCTCCCATGGACACAGTCACAGAGGC
TGGGATGGCCATAGCAATGGCGCTTACAGGCGGTATTGGCTTCATCCACCACAACTG
TACACCTGAATTCCAGGCCAATGAAGTTCGGAAAGTGAAGAAATATGAACAGGGAT
TCATCACAGACCCTGTGGTCCTCAGCCCCAAGGATCGCGTGCGGGATGTTTTTGAGG
CCAAGGCCCGGCATGGTTTCTGCGGTATCCCAATCACAGACACAGGCCGGATGGGG
AGCCGCTTGGTGGGCATCATCTCCTCCAGGGACATTGATTTTCTCAAAGAGGAGGAA
CATGACTGTTTCTTGGAAGAGATAATGACAAAGAGGGAAGACTTGGTGGTAGCCCC
TGCAGGCATCACACTGAAGGAGGCAAATGAAATTCTGCAGCGCAGCAAGAAGGGA
AAGTTGCCCATTGTAAATGAAGATGATGAGCTTGTGGCCATCATTGCCCGGACAGAC
CTGAAGAAGAATCGGGACTACCCACTAGCCTCCAAAGATGCCAAGAAACAGCTGCT
GTGTGGGGCAGCCATTGGCACTCATGAGGATGACAAGTATAGGCTGGACTTGCTCG
CCCAGGCTGGTGTGGATGTAGTGGTTTTGGACTCTTCCCAGGGAAATTCCATCTTCC
AGATCAATATGATCAAGTACATCAAAGACAAATACCCTAATCTCCAAGTCATTGGA
GGCAATGTGGTCACTGCTGCCCAGGCCAAGAACCTCATTGATGCAGGTGTGGATGCC
CTGCGGGTGGGCATGGGAAGTGGCTCCATCTGCATTATCCAGGAAGTGCTGGCCTGT
GGGCGGCCCCAAGCAACAGCAGTGTACAAGGTGTACGAGTATGCACGGCGCTTTGG
TGTTCCGGTCATTGCTGATGGAGGAATCCAAAATGTGGGTCATATTGCGAAAGCCTT
GGCCCTTGGGGCCTCCACAGTCATGATGGGCTCTCTCCTGGCTGCCACCACTGAGGC
CCCTGGTGAATACTTCTTTTCCGATGGGATCCGGCTAAAGAAATATCGCGGTATGGG
TTCTCTCGATGCCATGGACAAGCACCTCAGCAGCCAGAACAGATATTTCAGTGAAGC
TGACAAAATCAAAGTGGCCCAGGGAGTGTCTGGTGCTGTGCAGGACAAAGGGTCAA
TCCACAAATTTGTCCCTTACCTGATTGCTGGCATCCAACACTCATGCCAGGACATTG
GTGCCAAGAGCTTGACCCAAGTCCGAGCCATGATGTACTCTGGGGAGCTTAAGTTTG
AGAAGAGAACGTCCTCAGCCCAGGTGGAAGGTGGCGTCCATAGCCTCCATTCGTAT
GAGAAGCGGCTTTTCTGA
M-IMPDH2R; T333I, S351Y codon optimized CDS (SEQ ID NO: 4):
ATGGCCGATTACCTGATCTCCGGCGGCACCTCTTATGTGCCCGACGATGGCCTGACA
GCCCAGCAGCTGTTTAACTGTGGCGACGGCCTGACCTACAATGATTTCCTGATCCTG
CCTGGCTATATCGACTTTACAGCCGACCAGGTGGATCTGACCAGCGCCCTGACAAAG
AAGATCACCCTGAAGACACCACTGGTGAGCTCCCCTATGGACACCGTGACAGAGGC
CGGCATGGCCATCGCTATGGCCCTGACCGGCGGCATCGGCTTCATCCACCACAACTG
CACACCAGAGTTTCAGGCCAATGAGGTGAGAAAGGTGAAGAAGTACGAGCAGGGCT
TCATCACCGACCCTGTGGTGCTGAGCCCAAAGGACAGGGTGCGCGACGTGTTCGAG
GCCAAGGCCAGGCACGGCTTTTGCGGCATCCCCATCACCGATACAGGCCGGATGGG
CTCCAGACTGGTGGGCATCATCTCTAGCAGGGACATCGATTTCCTGAAGGAGGAGG
AGCACGACTGTTTTCTGGAGGAGATCATGACCAAGAGGGAGGATCTGGTGGTGGCA
CCTGCAGGCATCACACTGAAGGAGGCCAACGAGATCCTGCAGCGGTCTAAGAAGGG
CAAGCTGCCAATCGTGAATGAGGACGATGAGCTGGTGGCCATCATCGCCCGGACCG
ACCTGAAGAAGAACAGAGATTACCCTCTGGCCAGCAAGGACGCCAAGAAGCAGCTG
CTGTGCGGAGCAGCAATCGGCACACACGAGGACGATAAGTATCGGCTGGATCTGCT
GGCCCAGGCAGGAGTGGACGTGGTGGTGCTGGATTCCTCTCAGGGCAACAGCATCT
TCCAGATCAATATGATCAAGTACATCAAGGACAAGTATCCAAACCTGCAGGTCATC
GGAGGAAATGTGGTGACCGCAGCACAGGCCAAGAACCTGATCGACGCAGGAGTGG
ATGCACTGAGGGTGGGCATGGGCTCCGGCTCTATCTGCATCATCCAGGAGGTGCTGG
CCTGTGGCAGACCACAGGCAACCGCCGTGTATAAGGTGTACGAGTATGCCCGGAGA
TTTGGCGTGCCCGTGATCGCAGACGGAGGCATCCAGAATGTGGGACACATCGCAAA
GGCCCTGGCCCTGGGCGCCTCTACAGTGATGATGGGCAGCCTGCTGGCCGCAACCA
CAGAGGCACCAGGCGAGTACTTCTTTTCCGATGGCATCAGGCTGAAGAAGTATCGC
GGCATGGGCTCTCTGGACGCTATGGACAAGCACCTGAGCTCCCAGAATCGCTACTTC
TCCGAGGCCGACAAGATCAAGGTGGCACAGGGCGTGAGCGGAGCAGTGCAGGATA
AGGGCTCCATCCACAAGTTTGTGCCTTACCTGATCGCCGGCATCCAGCACTCTTGTC
AGGACATCGGAGCAAAGAGCCTGACCCAGGTGAGGGCCATGATGTATAGCGGCGAG
CTGAAGTTCGAGAAGCGCACATCTAGCGCCCAGGTGGAGGGAGGAGTGCACTCTCT
GCACAGCTACGAGAAGCGGCTGTTTTGA
Mutant CnB amino acid sequence (SEQ ID NO: 5):
MGNEASYPLEMCSHFDADEIKRLGKRFKKLDLDNSGSLSVEEFMSLPELQQNPLVQRVIDIFDTD
GNGEVDFKEFIEGVSQFSVKGDKEQKLRFAFRIYDMDKDGYISNGELFQVLKMMVGNNTKLAD
TQLQQIVDKTIINADKDGDGRISFEEFCAVVGGLDIHKKMVVDV
Mutant IMPDH amino acid sequence (SEQ ID NO: 6):
MADYLISGGTSYVPDDGLTAQQLFNCGDGLTYNDFLILPGYIDFTADQVDLTSALTKKITLKTPL
VSSPMDTVTEAGMAIAMALTGGIGFIHHNCTPEFQANEVRKVKKYEQGFITDPVVLSPKDRVRD
VFEAKARHGFCGIPITDTGRMGSRLVGIISSRDIDFLKEEEHDCFLEEIMTKREDLVVAPAGITL
KEANEILQRSKKGKLPIVNEDDELVAIIARTDLKKNRDYPLASKDAKKQLLCGAAIGTHEDDKYR
LDLLAQAGVDVVVLDSSQGNSIFQINMIKYIKDKYPNLQVIGGNVVTAAQAKNLIDAGVDALRVG
MGSGSICIIQEVLACGRPQATAVYKVYEYARRFGVPVIADGGIQNVGHIAKALALGASTVMMGS
LLAATTEAPGEYFFSDGIRLKKYRGMGSLDAMDKHLSSQNRYFSEADKIKVAQGVSGAVQDKG
SIHKFVPYLIAGIQHSCQDIGAKSLTQVRAMMYSGELKFEKRTSSAQVEGGVHSLHSYEKRLF

1. A modified T cell comprising an exogenous inhibitor of an immunosuppressant and an exogenous T-cell receptor (TCR).

2. The modified T cell of claim 1, comprising mRNA encoding the exogenous inhibitor of an immunosuppressant and mRNA encoding the exogenous T-cell receptor (TCR).

3. The modified T cell of claim 1, wherein the immunosuppressant is selected from Tacrolimus, Mycophenolate mofetil (MMF), or a combination thereof.

4. The modified T cell of claim 1, wherein the exogenous inhibitor is a mutant calcineurin (CN) subunit B (CnB) protein or a mutant inosine 5′-monophosphate dehydrogenase (IMPDH) protein.

5. (canceled)

6. The modified T cell of claim 4, wherein the mutant CnB comprises the amino acid sequence of SEQ ID NO:5 or an amino acid sequence having at least 90% sequence identity to SEQ ID NO:5; or
wherein the mutant IMPDH protein comprises the amino acid sequence of SEQ ID NO:6, or an amino acid sequence having at least 90% sequence identity to SEQ ID NO:6.

7. (canceled)

8. (canceled)

9. The modified T cell of claim 1, wherein the TCR specifically binds to a viral antigen selected from a hepatitis B virus (HBV) antigen, a CMV antigen, an EBV antigen, and influenza antigen, or a SARS antigen; or
wherein the TCR specifically binds to a viral antigen in Table 1.

10. (canceled)

11. (canceled)

12. The modified T cell of claim 1, wherein the T cell is isolated from a subject.

13. The modified T cell of claim 12, wherein the subject has a liver disease, has received an organ transplant or a stem cell transplant and is administered an immunosuppressant, has a viral infection or a tumor, and/or is immunocompromised.

14-16. (canceled)

17. A method for producing a modified T cell, comprising introducing an mRNA encoding an exogenous inhibitor of an immunosuppressant and an mRNA encoding an exogenous TCR into the T cell.

18. The method of claim 17, wherein the exogenous inhibitor is a mutant calcineurin (CN) subunit B (CnB) protein or a mutant IMIPDH protein.

19. The method of claim 18, wherein the mutant CnB comprises the amino acid sequence of SEQ ID NO:5 or an amino acid sequence having at least 90% sequence identity to SEQ ID NO:5; or
wherein the mutant IMPDH protein comprises the amino acid sequence of SEQ ID NO:6, or an amino acid sequence having at least 90% sequence identity to SEQ ID NO:6.

20. (canceled)

21. (canceled)

22. A method of treating a liver disease in a subject who has been administered an immunosuppressant, comprising introducing the T cell of claim 1 into the subject.

23-25. (canceled)

26. The method of claim 22, wherein the subject has a viral infection or has previously received an organ transplant or a stem cell transplant.

27. (canceled)

28. (canceled)

29. The method of claim 22, wherein the T cell is an autologous T cell.

30. The method of claim 22, wherein the immunosuppressant is Tacrolimus, Mycophenolate mofetil (MMF), or a combination thereof.

31. A method of treating liver disease in a subject in need thereof, comprising introducing mRNA into a T cell isolated from the subject, wherein the mRNA encodes a mutant CnB protein, a mutant IMPDH protein, or both, and mRNA encoding an exogenous T-cell receptor, and reintroducing the T cell into the subject, wherein the subject is administered an immunosuppressant.

32. (canceled)

33. (canceled)

34. The method of claim 31, wherein the subject has previously received a liver transplant.

35. The method of claim 31, wherein the immunosuppressant is selected from Tacrolimus, Mycophenolate mofetil (MMF), or a combination thereof.

36. The method of claim 31, wherein the exogenous T-cell receptor specifically binds to a viral antigen selected from a hepatitis B virus (HBV) antigen, a CMV antigen, or an EBV antigen; or
wherein the exogenous T-cell receptor specifically binds to an antigen in Table 1.

37. (canceled)

38. (canceled)

39. The method of claim 31, wherein the mutant CnB comprises the amino acid sequence of SEQ ID NO:5 or an amino acid sequence having at least 90% sequence identity to SEQ ID NO:5; or
wherein the mutant IMPDH protein comprises the amino acid sequence of SEQ ID NO:6, or an amino acid sequence having at least 90% sequence identity to SEQ ID NO:6.

40. (canceled)


SEQUENCE LISTING (70 sequences)

SEQ ID NO: 1 - DNA, 519 bp, Artificial Sequence
  Description of Artificial Sequence: Synthetic polynucleotide
atgggcaacgaggccagctaccctctggagatgtgctcccacttcgacgccgacgagatcaagcggctgggcaagcgcttcaagaagctggacctggacaacagcggcagcctgagcgtggaggagtttatgtctctgcccgagctgcagcagaaccccctggtgcagcgcgtgatcgacatcttcgacaccgacggcaacggcgaggtggacttcaaggagttcatcgagggcgtgagccagttcagcgtgaagggcgacaaggagcagaagctgcggttcgccttccggatctacgatatggataaagatggctatatttctaatggcgagctgttccaggtgctgaagatgatggtgggcaacaataccaagctggccgatacccagctgcagcagatcgtggacaagaccatcatcaacgccgacaaggacggcgacggcagaatcagcttcgaggagttctgtgccgtggtgggaggcctggatattcacaaaaaaatggtggtggacgtgtga

SEQ ID NO: 2 - DNA, 519 bp, Artificial Sequence
  Description of Artificial Sequence: Synthetic polynucleotide
atggggaatgaggcatcctatccactggaaatgtgcagccacttcgacgccgacgaaatcaaaagactggggaaaaggttcaaaaagctggacctggataacagcggctccctgtctgtggaggagttcatgtccctgcccgagctgcagcagaaccctctggtgcagagagtgatcgacatctttgacaccgatggcaatggcgaggtggatttcaaggagtttatcgagggcgtgagccagttctccgtgaagggcgacaaggagcagaagctgcggttcgcctttagaatctacgacatggataaggacggctatatctctaacggcgagctgtttcaggtgctgaagatgatggtgggcaacaataccaagctggccgacacacagctgcagcagatcgtggataagacaatcatcaatgccgataaggacggcgatggccggatcagcttcgaggagttctgtgccgtggtcggagggctggatattcataaaaagatggtcgtcgatgtctga

SEQ ID NO: 3 - DNA, 1545 bp, Artificial Sequence
  Description of Artificial Sequence: Synthetic polynucleotide
atggccgactacctgattagtgggggcacgtcctacgtgccagacgacggactcacagcacagcagctcttcaactgcggagacggcctcacctacaatgactttctcattctccctgggtacatcgacttcactgcagaccaggtggacctgacttctgctctgaccaagaaaatcactcttaagaccccactggtttcctctcccatggacacagtcacagaggctgggatggccatagcaatggcgcttacaggcggtattggcttcatccaccacaactgtacacctgaattccaggccaatgaagttcggaaagtgaagaaatatgaacagggattcatcacagaccctgtggtcctcagccccaaggatcgcgtgcgggatgtttttgaggccaaggcccggcatggtttctgcggtatcccaatcacagacacaggccggatggggagccgcttggtgggcatcatctcctccagggacattgattttctcaaagaggaggaacatgactgtttcttggaagagataatgacaaagagggaagacttggtggtagcccctgcaggcatcacactgaaggaggcaaatgaaattctgcagcgcagcaagaagggaaagttgcccattgtaaatgaagatgatgagcttgtggccatcattgcccggacagacctgaagaagaatcgggactacccactagcctccaaagatgccaagaaacagctgctgtgtggggcagccattggcactcatgaggatgacaagtataggctggacttgctcgcccaggctggtgtggatgtagtggttttggactcttcccagggaaattccatcttccagatcaatatgatcaagtacatcaaagacaaataccctaatctccaagtcattggaggcaatgtggtcactgctgcccaggccaagaacctcattgatgcaggtgtggatgccctgcgggtgggcatgggaagtggctccatctgcattatccaggaagtgctggcctgtgggcggccccaagcaacagcagtgtacaaggtgtacgagtatgcacggcgctttggtgttccggtcattgctgatggaggaatccaaaatgtgggtcatattgcgaaagccttggcccttggggcctccacagtcatgatgggctctctcctggctgccaccactgaggcccctggtgaatacttcttttccgatgggatccggctaaagaaatatcgcggtatgggttctctcgatgccatggacaagcacctcagcagccagaacagatatttcagtgaagctgacaaaatcaaagtggcccagggagtgtctggtgctgtgcaggacaaagggtcaatccacaaatttgtcccttacctgattgctggcatccaacactcatgccaggacattggtgccaagagcttgacccaagtccgagccatgatgtactctggggagcttaagtttgagaagagaacgtcctcagcccaggtggaaggtggcgtccatagcctccattcgtatgagaagcggcttttctga

SEQ ID NO: 4 - DNA, 1545 bp, Artificial Sequence
  Description of Artificial Sequence: Synthetic polynucleotide
atggccgattacctgatctccggcggcacctcttatgtgcccgacgatggcctgacagcccagcagctgtttaactgtggcgacggcctgacctacaatgatttcctgatcctgcctggctatatcgactttacagccgaccaggtggatctgaccagcgccctgacaaagaagatcaccctgaagacaccactggtgagctcccctatggacaccgtgacagaggccggcatggccatcgctatggccctgaccggcggcatcggcttcatccaccacaactgcacaccagagtttcaggccaatgaggtgagaaaggtgaagaagtacgagcagggcttcatcaccgaccctgtggtgctgagcccaaaggacagggtgcgcgacgtgttcgaggccaaggccaggcacggcttttgcggcatccccatcaccgatacaggccggatgggctccagactggtgggcatcatctctagcagggacatcgatttcctgaaggaggaggagcacgactgttttctggaggagatcatgaccaagagggaggatctggtggtggcacctgcaggcatcacactgaaggaggccaacgagatcctgcagcggtctaagaagggcaagctgccaatcgtgaatgaggacgatgagctggtggccatcatcgcccggaccgacctgaagaagaacagagattaccctctggccagcaaggacgccaagaagcagctgctgtgcggagcagcaatcggcacacacgaggacgataagtatcggctggatctgctggcccaggcaggagtggacgtggtggtgctggattcctctcagggcaacagcatcttccagatcaatatgatcaagtacatcaaggacaagtatccaaacctgcaggtcatcggaggaaatgtggtgaccgcagcacaggccaagaacctgatcgacgcaggagtggatgcactgagggtgggcatgggctccggctctatctgcatcatccaggaggtgctggcctgtggcagaccacaggcaaccgccgtgtataaggtgtacgagtatgcccggagatttggcgtgcccgtgatcgcagacggaggcatccagaatgtgggacacatcgcaaaggccctggccctgggcgcctctacagtgatgatgggcagcctgctggccgcaaccacagaggcaccaggcgagtacttcttttccgatggcatcaggctgaagaagtatcgcggcatgggctctctggacgctatggacaagcacctgagctcccagaatcgctacttctccgaggccgacaagatcaaggtggcacagggcgtgagcggagcagtgcaggataagggctccatccacaagtttgtgccttacctgatcgccggcatccagcactcttgtcaggacatcggagcaaagagcctgacccaggtgagggccatgatgtatagcggcgagctgaagttcgagaagcgcacatctagcgcccaggtggagggaggagtgcactctctgcacagctacgagaagcggctgttttga

SEQ ID NO: 5 - protein, 172 aa, Artificial Sequence
  Description of Artificial Sequence: Synthetic polypeptide
MGNEASYPLEMCSHFDADEIKRLGKRFKKLDLDNSGSLSVEEFMSLPELQQNPLVQRVIDIFDTDGNGEVDFKEFIEGVSQFSVKGDKEQKLRFAFRIYDMDKDGYISNGELFQVLKMMVGNNTKLADTQLQQIVDKTIINADKDGDGRISFEEFCAVVGGLDIHKKMVVDV

SEQ ID NO: 6 - protein, 514 aa, Artificial Sequence
  Description of Artificial Sequence: Synthetic polypeptide
MADYLISGGTSYVPDDGLTAQQLFNCGDGLTYNDFLILPGYIDFTADQVDLTSALTKKITLKTPLVSSPMDTVTEAGMAIAMALTGGIGFIHHNCTPEFQANEVRKVKKYEQGFITDPVVLSPKDRVRDVFEAKARHGFCGIPITDTGRMGSRLVGIISSRDIDFLKEEEHDCFLEEIMTKREDLVVAPAGITLKEANEILQRSKKGKLPIVNEDDELVAIIARTDLKKNRDYPLASKDAKKQLLCGAAIGTHEDDKYRLDLLAQAGVDVVVLDSSQGNSIFQINMIKYIKDKYPNLQVIGGNVVTAAQAKNLIDAGVDALRVGMGSGSICIIQEVLACGRPQATAVYKVYEYARRFGVPVIADGGIQNVGHIAKALALGASTVMMGSLLAATTEAPGEYFFSDGIRLKKYRGMGSLDAMDKHLSSQNRYFSEADKIKVAQGVSGAVQDKGSIHKFVPYLIAGIQHSCQDIGAKSLTQVRAMMYSGELKFEKRTSSAQVEGGVHSLHSYEKRLF

SEQ ID NO: 7-24 - skipped (18 entries)

SEQ ID NO: 25 - protein, 10 aa, Hepatitis B virus
FLPSDFFPSV

SEQ ID NO: 26-55 - skipped (30 entries)

SEQ ID NO: 56 - protein, 10 aa, Hepatitis B virus
SIVSPFIPLL

SEQ ID NO: 57-58 - skipped (2 entries)

SEQ ID NO: 59 - protein, 9 aa, Human betaherpesvirus 5
KEVNSQLSL

SEQ ID NO: 60 - protein, 9 aa, Human betaherpesvirus 5
ATVQGQNLK

SEQ ID NO: 61 - protein, 9 aa, Human betaherpesvirus 5
NLVPMVATV

SEQ ID NO: 62 - protein, 10 aa, Human gammaherpesvirus 4
AVFDRKSDAK

SEQ ID NO: 63 - protein, 10 aa, Hepatitis B virus
FLGPLLVLQA

SEQ ID NO: 64 - protein, 9 aa, Hepatitis B virus
FLLTRILTI

SEQ ID NO: 65 - protein, 10 aa, Severe acute respiratory syndrome-related coronavirus
GETALALLLL

SEQ ID NO: 66 - protein, 9 aa, Influenza virus
GILGFVFTL

SEQ ID NO: 67 - RNA, 19 nt, Unknown
  Description of Unknown: FKBP1A siRNA sequence
gagccaaacugacuauauc

SEQ ID NO: 68 - RNA, 19 nt, Unknown
  Description of Unknown: FKBP1A siRNA sequence
gacagaaacaagcccuuua

SEQ ID NO: 69 - RNA, 19 nt, Unknown
  Description of Unknown: FKBP1A siRNA sequence
aaacuggaaugacaggaau

SEQ ID NO: 70 - RNA, 19 nt, Unknown
  Description of Unknown: FKBP1A siRNA sequence
gaaauuugauuccucccgg